(19) United States
(12) Patent Application Publication
(10) Pub. No.: US 2025/0108211 A1
(43) Pub. Date: Apr. 3, 2025

(54) APPARATUS AND METHOD OF REAPPLYING TRANSDUCER ARRAYS TO A PATIENT

(71) Applicant: Novocure GmbH, Root D4 (CH)

(72) Inventor: Amit Segal, Karmiel (IL)

(21) Appl. No.: 18/896,044

(22) Filed: Sep. 25, 2024

Related U.S. Application Data
(60) Provisional application No. 63/586,710, filed on Sep. 29, 2023.
(60) Provisional application No. 63/609,693, filed on Dec. 13, 2023.

(51) Int. Cl. A61N 1/36; A61N 1/04

(52) U.S. Cl. CPC A61N 1/36002; A61N 1/0476; A61N 1/0484

(57) ABSTRACT

Systems, apparatuses, and methods for applying TTFields are disclosed, including an apparatus comprising a frame having a bodily interface for engaging a patient's body part, a support panel for covering a portion of a treatment area, a transducer array having a skin-facing surface and an opposed support panel surface and comprising a complementary array fastener positioned on the opposed support panel surface and an attaching component positioned on the skin-facing surface, and an array fastener connected to the support panel for attaching to the complementary array fastener to support the transducer array within the portion of the treatment area, wherein the array fastener is separable from the complementary array fastener with a first force, the attaching component is separable from the patient with a second force, and applying the first force to the transducer array in a direction away from the patient does not cause separation.

### REFERENCE TO RELATED APPLICATIONS

The present patent application claims priority to the provisional patent application identified by U.S. Ser. No. 63/586,710, and filed on Sep. 29, 2004. The present patent application also claims priority to the provisional patent application identified by 63/609,693 and filed on Dec. 13, 2023. The disclosures of both provisional patent applications are hereby incorporated herein by reference.

### BACKGROUND ART

TTFields therapy is a proven approach for treating tumors. For example, using the Optune® system for delivering tumor treating fields (i.e., TTFields), the TTFields are delivered to patients via four transducer arrays placed on the patient's skin in close proximity to a tumor. The transducer arrays are arranged in two pairs, and each transducer array is connected via a multi-wire cable to an electric field generator. The electric field generator (a) sends an AC current through one pair of arrays during a first period of time; then (b) sends an AC current through the other pair of arrays during a second period of time; then repeats steps (a) and (b) for the duration of the treatment.

During an initial consultation, patients are generally assisted by a doctor, nurse, caregiver, or therapist in determining locations for each of the transducer arrays on the patients' bodies based on the size, location, and characteristics of a tumor and applying the transducer arrays on the patients' bodies in said locations. However, patients may encounter difficulty when attempting to reapply and maintain the transducer arrays on their own bodies, particularly by hand and without the assistance of a doctor, nurse, caregiver, or therapist. Nevertheless, some patients are unable or disinclined to rely on others to assist in their care. Further, in order to effectively deliver the TTFields with the transducer arrays, it is important that the transducer arrays are reapplied and maintained at the specific, predetermined locations on the patient's body, further adding to the difficulty encountered by patients.

### SUMMARY OF THE INVENTION

It is desirable for a system and method to assist a patient in reapplying and maintaining the transducer arrays at predetermined locations on the patient's body. It is to such a system and method that the present disclosure is directed.

In some embodiments, the problem of assisting the patient in reapplying the transducer arrays and maintaining a sufficient electrical connection between the transducer arrays and the patient's body at predetermined locations on the patient is solved by an apparatus, comprising: a frame having one or more bodily interface configured to engage a body part of a patient and one or more support panel fastener; a support panel sized and dimensioned to cover at least a portion of a treatment area of a target region and comprising one or more complementary support panel fastener configured to attach to the one or more support panel fastener; a transducer array having a skin-facing surface and an opposed support panel surface and comprising a complementary transducer array fastener positioned on the opposed support panel surface and an attaching component positioned on the skin-facing surface; and one or more transducer array fastener connected to the support panel and configured to attach to the complementary transducer array fastener so as to support the transducer array within at least the portion of the treatment area, wherein the one or more transducer array fastener is separable from the complementary transducer array fastener with a first force, the attaching component when attached to the patient is separable from the patient with a second force greater than the first force, and application of the first force to the transducer array in a direction away from the patient does not cause separation of the transducer array from the patient.

The details of one or more embodiments of the subject matter described in this specification are set forth in the accompanying drawings and the description below. Other aspects, features, and advantages of the subject matter will become apparent from the description, the drawings, and the claims.

### BRIEF DESCRIPTION OF THE DRAWINGS

The accompanying drawings, which are incorporated in and constitute a part of this specification, illustrate one or more embodiments described herein and, together with the description, explain these embodiments. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a diagrammatic view of an exemplary embodiment of a system for applying TTFields to a patient constructed in accordance with the present disclosure;

FIG. 2 is a perspective view of an exemplary embodiment of an apparatus for use in the system shown in FIG. 1, wherein the apparatus is configured to be worn on a patient's head;

FIG. 3A is a perspective view of an exemplary embodiment of a frame of the apparatus shown in FIG. 2;

FIG. 3B is a top view of the frame shown in FIG. 3A;

FIG. 3C is a left side view of the frame shown in FIG. 3A;

FIG. 3D is a front view of the frame shown in FIG. 3A;

FIG. 4A is a perspective view of an exemplary embodiment of a support panel of the apparatus shown in FIG. 2;

FIG. 4B is a perspective view of another exemplary embodiment of the support panel of the apparatus shown in FIG. 2;

FIG. 5 is a partial cross-sectional top view of an exemplary embodiment of the support panel receiving a transducer array constructed in accordance with the present disclosure;

FIG. 6 is a process flow diagram of a method for applying TTFields to a patient in accordance with the present disclosure;

FIG. 7A is a front view of another exemplary embodiment of the apparatus shown in FIG. 2, wherein the apparatus is configured to be worn on a patient's torso;

FIG. 7B is a back view of the apparatus shown in FIG. 7A;

FIG. 7C is a right side view of the apparatus shown in FIG. 7A;

FIG. 8A is a front view of an exemplary embodiment of a frame of the apparatus shown in FIG. 7A;

FIG. 8B is a back view of the frame shown in FIG. 8A;

FIG. 8C is a right side view of the frame shown in FIG. 8A;

FIG. 8D is a left side view of the frame shown in FIG. 8A; and

FIG. 9 is a process flow diagram of another method for applying TTFields to a patient in accordance with the present disclosure.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

Headings are provided for convenience only and are not to be construed to limit the invention in any manner. Embodiments illustrated under any heading or in any portion of the disclosure may be combined with embodiments illustrated under the same or any other heading or other portion of the disclosure. Any combination of the elements described herein in all possible variations thereof is encompassed by the disclosure unless otherwise indicated herein or otherwise clearly contradicted by context.

As utilized in accordance with the present disclosure, the following terms, unless otherwise indicated, shall be understood to have the following meanings.

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, or greater numbers of compounds. The term “plurality” refers to “two or more.”

The use of the term “at least one” will be understood to include one as well as any quantity more than one. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “patient” as used herein encompasses any mammals including human and veterinary subjects. “Mammal” for purposes of treatment refers to any animal classified as a mammal, including (but not limited to) humans, domestic and farm animals, nonhuman primates, and any other animal that has mammary tissue. In some embodiments, the term “patient” may apply to a simulation mannequin for use in teaching, for example.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), a field programmable gate array (FPGA), a combination of hardware and software, and/or the like. The term “processor” as used herein means a single processor or multiple processors working independently or together to perform a task.

The term “TTField”, as used herein, means tumor treating field. TTFields are low intensity (e.g., 1-4 V/cm) alternating electric fields of medium frequencies (about 50 kHz to about 1 MHz, and more preferable from about 50 kHz to about 500 kHz) that when applied to a conductive medium, such as a human body, via electrodes may be used, for example, to treat tumors as described in U.S. Pat. Nos. 7,016,725, 7,089,054, 7,333,852, 7,565,205, 7,805,201, and 8,244,345 by Palti and in the publication “Disruption of Cancer Cell Replication by Alternating Electric Fields”, Cancer Res. 2004 64:3288-3295, Eilon D. Kirson, et al., all of which are hereby expressly incorporated by reference herein in their entirety. TTFields have been shown to have the capability to specifically affect cancer cells and serve, among other uses, for treating cancer. TTFields therapy is an approved mono-treatment for recurrent glioblastoma (GBM), and an approved combination therapy with chemotherapy for newly diagnosed GBM patients.

The term “transducer array”, as used herein, means a conductive transducer array or a non-conductive transducer array. Exemplary transducer arrays may include, for example, pads disclosed in any one of U.S. Patent Publication No. 2021/0346693 entitled “CONDUCTIVE PAD GENERATING TUMOR TREATING FIELD AND METHODS OF PRODUCTION AND USE THEREOF” and U.S. Patent Application No. 63/128,265 entitled “OPTIMIZATION OF COMPOSITE ELECTRODE”, all of which are hereby expressly incorporated by reference herein in their entirety.

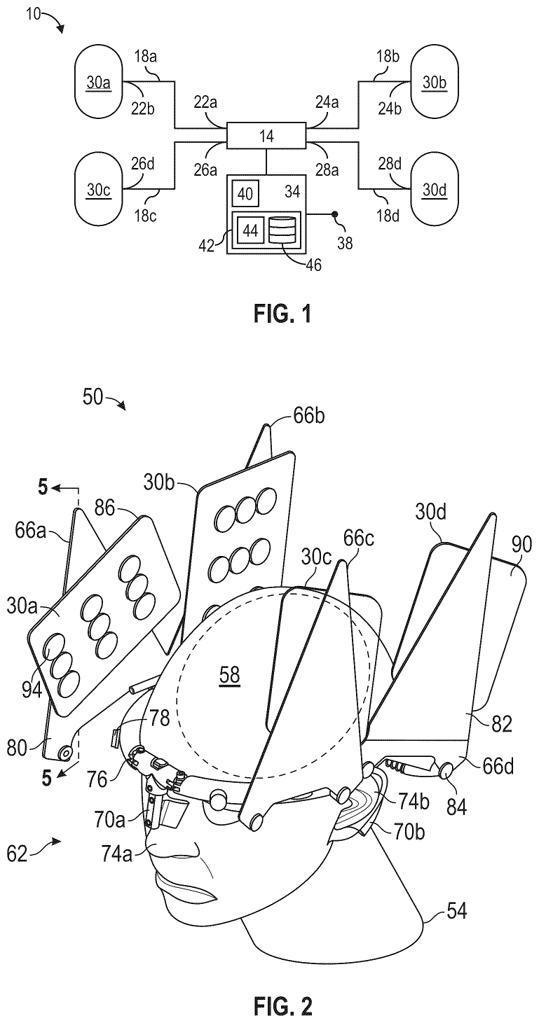

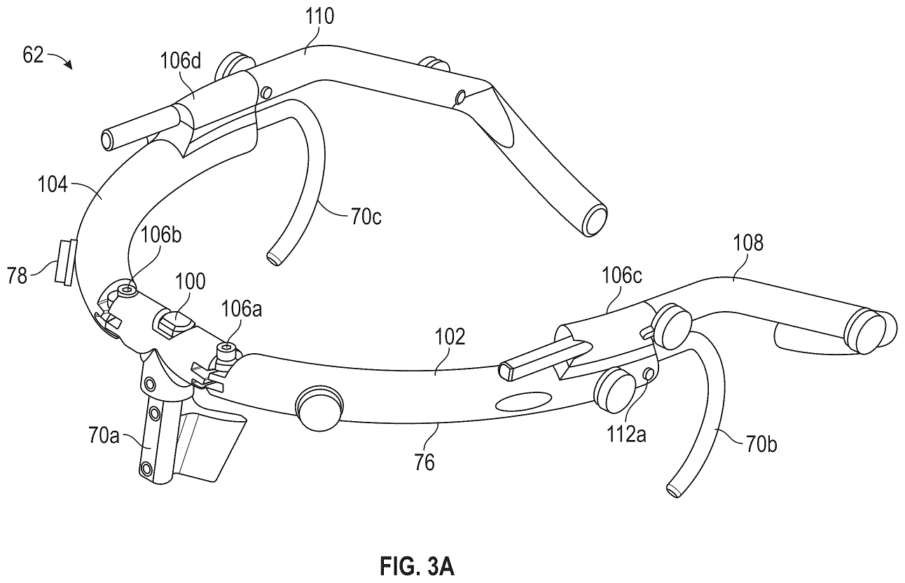

Referring now to the drawings and in particular to FIG. 1, shown therein is an exemplary embodiment of a system 10 for applying TTFields to a patient 54 (shown in FIG. 2) constructed in accordance with the present disclosure. The system 10 generally comprises an electric field generator 14 and a plurality of conductive leads, shown in FIG. 1 as a first conductive lead 18a, a second conductive lead 18b, a third conductive lead 18c, and a fourth conductive lead 18d (collectively, the “conductive leads 18”). While the system 10 is shown in FIG. 1 as comprises four of the conductive leads 18, in other embodiments, the system 10 comprises more or less than four of the conductive leads 18.

The electric field generator 14 is operable to supply power and generate an electrical signal (i.e., a TTField signal), which may be an alternating current electrical signal or a pulsed electrical signal having a frequency in a range from 50 kHz to 1 MHz (or, preferably, from 100 kHz to 500 kHz). A voltage of the TTField signal may be such that an electric field (i.e., a TTField) in tissue within a treatment area 58 (shown in FIG. 2) has an intensity in a range from 0.1 V/cm to 10 V/cm.

The first conductive lead 18a has a first end 22a and a second end 22b, the second conductive lead 18b has a first end 24a and a second end 24b, the third conductive lead 18c has a first end 26a and a second end 26b, and the fourth conductive lead 18d has a first end 28a and a second end 28b. The conductive leads 18 are isolated conductors with a flexible metal shield and are preferably grounded, thereby preventing spread of any electric field generated by the conductive leads 18.

Each of the conductive leads 18 are electrically connected to the electric field generator 14. Accordingly, the first end 22a of the first conductive lead 18a, the first end 24a of the second conductive lead 18b, the first end 26a of the third conductive lead 18c, and the first end 28a of the fourth conductive lead 18d are electrically connected to the electric field generator 14.

The conductive leads 18 are also electrically connected to a plurality of transducer arrays, shown in FIG. 1 as a first transducer array 30a, a second transducer array 30b, a third transducer array 30c, and a fourth transducer array 30d (collectively, the “transducer arrays 30”). Accordingly, the second end 22b of the first conductive lead 18a, the second end 24b of the second conductive lead 18b, the second end 26b of the third conductive lead 18c, and the second end 28b of the fourth conductive lead 18d are electrically connected to the first transducer array 30a, the second transducer array 30b, the third transducer array 30c, and the fourth transducer array 30d, respectively.

Each of the transducer arrays 30 is supplied with the TTField signals. The transducer arrays 30, being supplied with the TTField signals, cause an electrical current to flow between the transducer arrays 30, thereby causing the TTField to be generated between the transducer arrays 30. The transducer arrays 30, when applied to a treatment area 58 (shown in phantom in FIG. 2) on a patient 54 (also shown in FIG. 2), cause the TTField to be generated in a target region within the patient 54. The target region may include one or more tumor, and the generation of the TTField may selectively destroy and/or inhibit growth of the at least one tumor. While the system 10 is shown in FIG. 1 as comprising four of the transducer arrays 30, in other embodiments, the system 10 comprises more or less than four of the transducer arrays 30.

To optimize a distribution of the TTField, the transducer arrays 30 may be configured differently based on a particular application in which the transducer arrays 30 are used. Further, the transducer arrays 30 may have specific shapes and positions so as to generate the TTField having a desired configuration, direction, and/or intensity at the treatment area 58 and only at the treatment area 58 so as to focus the treatment in the target region. A user may apply the transducer arrays 30 to the treatment area 58 on the patient 54 (i.e., applied to the patient's skin) to cause the TTField to be generated within the target region. The TTField may be of a widely distributed type or a local type (e.g., to treat skin tumors or lesions close to the skin surface).

The user may be a medical professional, such as a doctor, nurse, caregiver, therapist, or other person acting under the instruction of a doctor, nurse, caregiver, or therapist. In some embodiments, the user may be the patient 54 (i.e., the patient 54 and/or a helper may reapply the transducer arrays 30 to the treatment area 58 in predetermined locations).

In some embodiments, the system 10 further comprises a controller 34. The controller 34 generally comprises circuitry operable to control an output of the electric field generator 14 (e.g., to set the output at a maximum value that does not cause excessive heating of the treatment area 58). The controller 34 may present the user with a warning or the like when a temperature of the treatment area 58 as sensed by one or more temperature sensor 38 (hereinafter, the “temperature sensor 38”), exceeds a predetermined threshold. In some embodiments, the controller 34 may deactivate the electric field generator 14 or decrease the power of the TTField signal generated by the electric field generator 14 if the temperature of the treatment area 58, as sensed by the temperature sensor 38, is above the predetermined threshold. The predetermined threshold may be a temperature at which the patient 54 could be made uncomfortable while using the system 10. For example, the predetermined threshold may be a temperature between 39 degrees Celsius and 42 degrees Celsius. The temperature sensor 38 may be connected to or otherwise associated with one or more of the transducer arrays 30 so as to sense the temperature of the treatment area 58 at one or more of the transducer arrays 30.

In some embodiments, the circuitry of the controller 34 includes a processor 40 and a non-transitory processor-readable medium 42 (hereinafter, the “memory 42”). The memory 42 may store processor-executable instructions 44 and/or a datastore 46. The processor-executable instructions 44 when executed by the processor 40 may cause the processor 40 to perform one or more action described herein. The processor 40 may be in communication with the temperature sensor 38 and/or other circuitry, such as an analog-to-digital converter, a digital-to-analog converter, a multimeter, an ohmmeter, a voltmeter, and/or an ammeter, for example.

Referring now to FIG. 2, shown therein is an exemplary embodiment of an apparatus 50 for use in implementing the system 10 shown in FIG. 1. The apparatus 50 may be temporarily worn by a patient 54 and may assist the patient 54 in placing and maintaining the transducer arrays 30 on the treatment area 58 in predetermined locations. The apparatus 50 generally comprises a frame 62 and one or more support panel, such as a first support panel 66a, a second support panel 66b, a third support panel 66c, and a fourth support panel 66d shown in FIG. 2 (collectively, the “support panels 66”). In some embodiments, each of the support panels 66 temporarily supports one or more of the transducer arrays 30 attached thereto.

In some embodiments, the apparatus 50 is configured to be worn on the head of the patient 54, as shown in FIG. 2. In other embodiments, however, the apparatus 50 may be configured to be worn on a different body part of the patient 54, such as one or more of a torso, arm, waist, leg, hand, or foot of the patient 54. For example, the apparatus 50 may be a torso covering (e.g., a vest), an arm covering (e.g., a sleeve), a hand covering (e.g., a glove), a foot covering (e.g., a sock or shoe), a leg or waist covering (e.g., pants, leggings, shorts, or stockings), and/or the like. Throughout this application, the apparatus 50 may be described as being a headpiece; however, the apparatus 50 is not limited to being a headpiece, as described above.

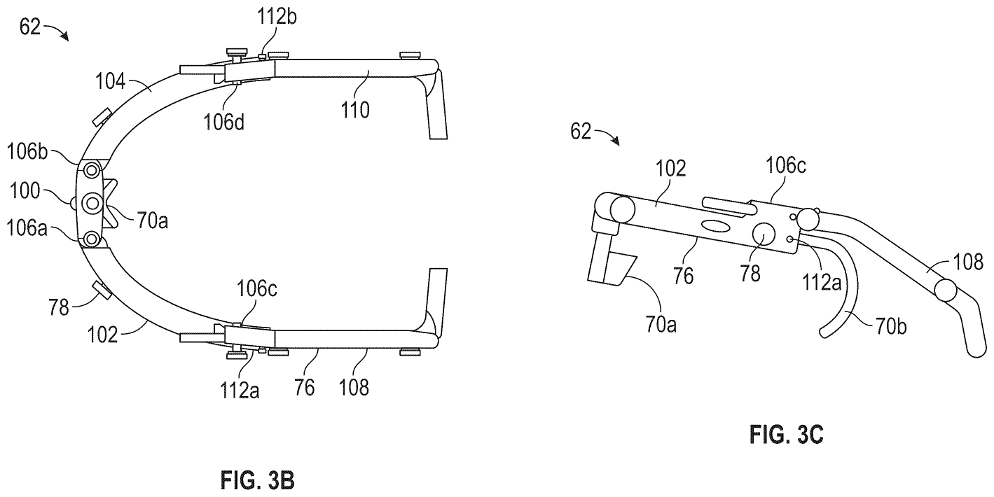

The frame 62 may have one or more bodily interface 70 (hereinafter the “bodily interfaces 70”), such as a nose interface 70a and a left ear interface 70b shown in FIG. 2 and a right ear interface 70c shown in FIG. 3A, configured to engage a body part of the patient 54, such as the patient's nose 74a, the patient's left ear 74b, and the patient's right ear (not shown), respectively. In some embodiments, the frame 62 may be rigid such that, when the bodily interfaces 70 are engaging the body parts of the patient 54, the frame 62 is maintained in a fixed position without substantial deflection when supporting the support panels 66.

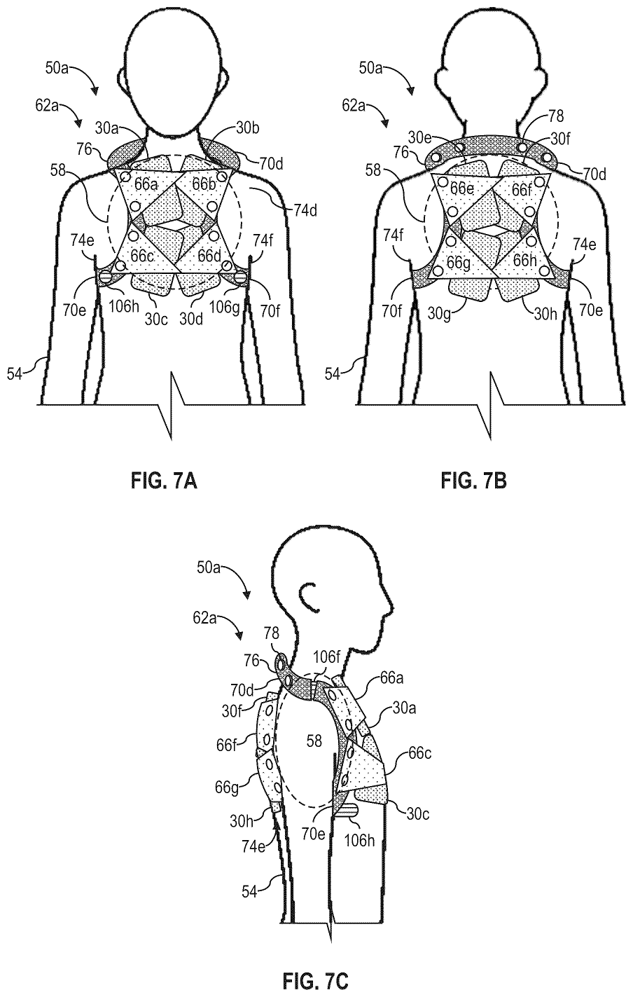

In embodiments in which the apparatus 50 is configured to be worn on a different body part of the patient 54 (i.e., a body part other than the patient's head), the bodily interfaces 70 may be configured to engage different body parts of the patient. For example, in embodiments in which the apparatus 50 is a torso covering (shown in FIGS. 7A-7C), the bodily interfaces 70 may include a shoulder interface 70d (shown in FIGS. 7A-7C) configured to engage a patient's shoulders 74d (shown in FIGS. 7A-7C).

The frame 62 may comprise an outer surface 76 and one or more support panel fastener 78 (hereinafter, the “support panel fasteners 78”) in one or more location on the outer surface 76 adjacent to the treatment area 58 when the apparatus 50 is positioned on the patient 54. For purposes of clarity, only one of the support panel fasteners 78 is labeled with a reference character. In the embodiment shown, the frame 62 includes eight of the support panel fasteners 78 spatially disposed on the outer surface 76 of the frame 62 and configured to be positioned adjacent to the treatment area 58 when the apparatus 50 is positioned on the patient 54.

Each of the support panels 66 may be sized and dimensioned to cover at least a portion of the treatment area 58 and may have a skin-facing surface 80 (collectively, the “skin-facing surfaces 80”) and an opposed outer surface 82 (collectively, the “opposed outer surfaces 82”). For purposes of clarity, only one of the skin-facing surfaces 80 and only one of the opposed outer surfaces 82 is labeled with a reference character. In some embodiments, the support panels 66 may be flexible or movable to cover at least the portion of the treatment area 58 when attached to the frame 62.

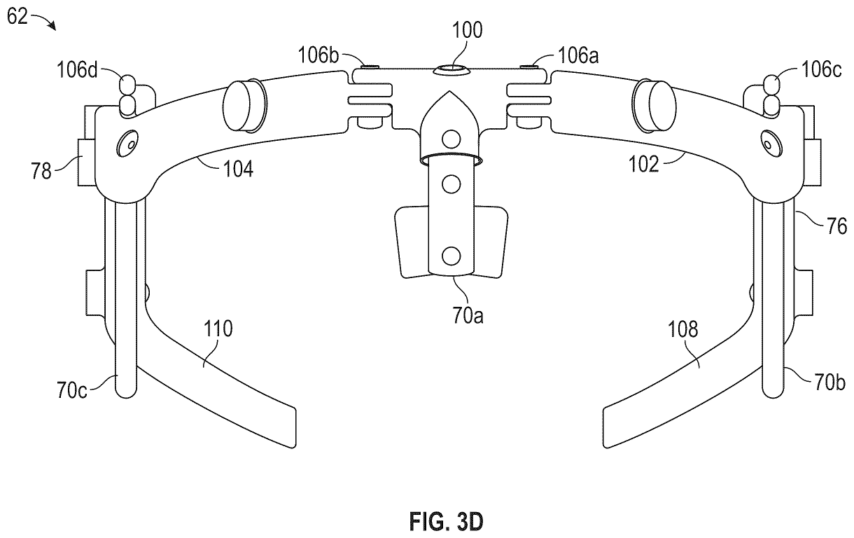

Each of the support panels 66 may further comprise one or more complementary support panel fastener 84 (hereinafter, the “complementary support panel fasteners 84”) positioned on the skin-facing surface 80 and one or more transducer array fastener 114 (hereinafter the “transducer array fasteners 114”) (shown in FIG. 4A). For purposes of clarity, only one of the complementary support panel fasteners 84 is labeled with a reference character. The support panels 66 may be at least somewhat flexible and/or bendable. The support panel fasteners 78 and the complementary support panel fasteners 84 may be configured to attach to one another to support the support panels 66 on the frame 62 such that the support panels 66 may be moved towards the patient 54 (e.g., by bending the support panels 66 inwards towards the treatment area 58) to apply the transducer arrays 30 to the treatment area 58.

Each of the transducer arrays 30 may have a skin-facing surface 86 (collectively, the “skin-facing surfaces 86”) and an opposed support panel surface 90 (collectively, the “opposed support panel surfaces 90”). For purposes of clarity, only one of the skin-facing surfaces 86 and only one of the opposed support panel surfaces 90 is labeled with a reference character. Each of the transducer arrays 30 may comprise one or more attaching component 94 (collectively, the “attaching components 94”) positioned on the skin-facing surface 86 and one or more complementary transducer array fastener 98 (hereinafter, the “complementary transducer array fasteners 98”) (shown in FIG. 4A) positioned on the opposed support panel surface 90. For purposes of clarity, only one of the attaching components 94 and only one of the complementary transducer array fasteners 98 is labeled with a reference character.

In some embodiments, a pair including one of the support panel fasteners 78 and a corresponding one of the complementary support panel fasteners 84 is referred to as a support panel fastener set. In some embodiments, each support panel fastener set is a snap fastener comprising a snap member and a receiving member (i.e., one of the support panel fastener 78 and the complementary support panel fastener 84 is the snap member and the other is the receiving member), wherein the snap member is configured to engage the receiving member. In other embodiments, each support panel fastener set is a complementary (e.g., hook-and-loop) fastener set having a first complementary fastener and a second complementary fastener. In embodiments wherein each support panel fastener set is a hook-and-loop fastener, one of the first complementary fastener and the second complementary fastener comprises a hook member and the other one of the first complementary fastener and the second complementary fastener comprises a loop member (i.e., one of the support panel fastener 78 and the complementary support panel fastener 84 is the hook member and the other is the loop member), wherein the hook member is configured to engage and be secured to the loop member.

Referring now to FIGS. 3A-3D, shown therein is an exemplary embodiment of the frame 62 of the apparatus 50 shown in FIG. 2. The frame 62 may comprise a central portion 100, a left portion 102, and a right portion 104 shown in FIG. 3A. The central portion 100 may have one or more connection point, such as a left connection point 106a and a right connection point 106b shown in FIG. 3A.

The left portion 102 may be connected to the central portion 100 at the left connection point 106a such that the left portion 102 is pivotable about the left connection point 106a, and the right portion 104 may be connected to the central portion 100 at the right connection point 106b such that the right portion 104 is pivotable about the right connection point 106b. As further shown in FIG. 3A, the nose interface 70a may be connected to and extend below the central portion 100, the left ear interface 70b may be connected to and extend below the left portion 102, and the right ear interface 70c configured to engage a patient's right ear may be connected to and extend below the right portion 104.

In some embodiments, the frame 62 further comprises a left rear portion 108 and a right rear portion 110. In such embodiments, the left portion 102 may have a left rear connection point 106c and the right portion 104 may have a right rear connection point 106d. The left rear portion 108 may slidingly engage the left rear connection point 106c such that the left rear portion 108 may slide forwards and backwards to accommodate different sizes of the patient 54. The right rear portion 110 may slidingly engage the right rear connection point 106d such that the right rear portion 110 may slide forwards and backwards to accommodate different sizes of the patient 54. In some embodiments, one or more of the left rear connection point 106c and the right rear connection point 106d may have one or more locking mechanism, such as a left locking mechanism 112a shown in FIGS. 3A-3C, and a right locking mechanism 112b shown in FIG. 3B, to maintain the left rear portion 108 and the right rear portion 110 in place when the left locking mechanism 112a and the right locking mechanism 112b are locked.

Although the embodiment of the frame 62 disclosed herein is constructed of different parts that are movable relative to each other to be able to adjust the size of the frame 62, it should be understood that the frame 62 may be formed as a unitary device designed to fit a particular size of the patient 54.

Referring now to FIG. 4A, shown therein is an exemplary embodiment of one of the support panels 66 shown in FIG. 2, shown as the first support panel 66a in FIG. 4A. The transducer array fasteners 114, shown as a first transducer array fastener 114a, a second transducer array fastener 114b, a third transducer array fastener 114c, and a fourth transducer array fastener 114d in FIG. 4A, may be connected to the first support panel 66a and may be configured to attach to one of the complementary transducer array fasteners 98, such as a first complementary transducer array fastener 98a, a second complementary transducer array fastener 98b, a third complementary transducer array fastener 98c, and a fourth complementary transducer array fastener 98d shown in FIG. 4A, of one of the transducer arrays 30, such as the first transducer array 30a shown in FIG. 4A, so as to support the first transducer array 30a within at least a portion of the treatment area 58. In this embodiment, the first transducer array 30a may be rotated 90 degrees in either direction (or 180 degrees) before being attached to the first support panel 66a.

Referring now to FIG. 4B, shown therein is another exemplary embodiment of one of the support panels 66 shown in FIG. 2, shown as the first support panel 66a in FIG. 4B. As shown in FIG. 4B, a single transducer array fastener 114 may be connected to the first support panel 66a and may be configured to attach to a single complementary transducer array fastener 98 so as to support the first transducer array 30a within at least a portion of the treatment area 58. In this embodiment, the first transducer array 30a may be rotated between 0 and 180 degrees in either direction before being attached to the first support panel 66a.

In some embodiments, a pair including one of the transducer array fasteners 114 and a corresponding one of the complementary transducer array fasteners 98 is referred to as a transducer array fastener set. In some embodiments, each transducer array fastener set is a snap fastener comprising a snap member and a receiving member (i.e., one of the transducer array fastener 114 and the complementary transducer array fastener 98 is the snap member and the other is the receiving member), wherein the snap member is configured to engage the receiving member. In other embodiments, each transducer array fastener set may be a hook-and-loop fastener set comprising a hook member and a loop member (i.e., one of the transducer array fastener 114 and the complementary transducer array fastener 98 is the hook member and the other is the loop member), wherein the hook member is configured to engage the loop member. The transducer array fasteners 114 and the corresponding ones of the complementary transducer array fasteners 98 may be constructed in other ways, such as complementary magnets.

By attaching the first transducer array 30a having the complementary transducer array fasteners 98 to the first support panel 66a having the transducer array fasteners 114, the first transducer array 30a may be supported within at least a first portion of the treatment area 58. Further, by attaching the second transducer array 30b having the complementary transducer array fasteners 98 to the second support panel 66b having the transducer array fasteners 114 (as shown in FIG. 2), the second transducer array 30b may be supported within at least a second portion of the treatment area 58. Further still, by attaching the third transducer array 30c having the complementary transducer array fasteners 98 to the third support panel 66c having the transducer array fasteners 114 (as shown in FIG. 2), the third transducer array 30c may be supported within at least a third portion of the treatment area 58. Finally, by attaching the fourth transducer array 30d having the complementary transducer array fasteners 98 to the fourth support panel 66d having the transducer array fasteners 114 (as shown in FIG. 2), the fourth transducer array 30d may be supported within at least a fourth portion of the treatment area 58.

Accordingly, the first transducer array 30a, the second transducer array 30b, the third transducer array 30c, and the fourth transducer array 30d may be supported within at least a first portion, a second portion, a third portion, and a fourth portion of the treatment area 58, respectively. In some embodiments, two or more of the first portion, the second portion, the third portion, and the fourth portion of the treatment area 58 partially coincide. In other embodiments, two or more of the first portion of the second portion of the third portion and the fourth portion of the treatment area 58 substantially coincide.

In some embodiments, two or more of the first portion, the second portion, the third portion, and the fourth portion of the treatment area 58 are selected to target the same target region within the patient 54. In other embodiments, two or more of the first portion, the second portion, the third portion, and the fourth portion of the treatment area 58 are selected to target different target regions within the patient 54.

In some embodiments, the transducer array fasteners 114 of each of the support panels 66 may be arranged in such a way as to attach to the complementary transducer array fasteners 98 of a particular one of the transducer arrays 30 when the particular one of the transducer arrays 30 is arranged in a recommended arrangement, which may be determined by a doctor, nurse, caregiver, or therapist in a consultation with the patient 54. That is, the transducer array fasteners 114 may be positioned on the skin-facing surface 80 (shown in FIG. 2) of the support panels 66 in such a way as to support the transducer arrays 30 only in the recommended arrangement to assist the patient 54 in reapplying the transducer arrays 30 to the support panels 66 and using the apparatus 50 and support panels 66 to reapply the transducer arrays 30 to the treatment area 58.

In some embodiments, the opposed support panel surfaces 90 of the transducer arrays 30 may have one or more reference point 116 (hereinafter the “reference points 116”) for attaching the complementary transducer array fasteners 98 thereto. For purposes of clarity, only one of the reference points 116 is labeled with a reference character. In some embodiments, the reference points 116 may have rigid surfaces which may assist in adhering the complementary transducer array fasteners 98 to the opposed support panel surfaces 90. In other embodiments, the reference points 116 may include markings or indicia to provide an indication for where the complementary transducer array fasteners 98 should be attached to attach the transducer arrays 30 to the support panels 66 in the recommended arrangement.

In use, the transducer arrays 30 may be applied to the patient in the recommended arrangement by a care giver, such as a doctor, nurse, therapist, or the like. Then, the apparatus 50 is positioned on the patient 54 with the support panels 66 connected to the frame 62. The complementary transducer array fasteners 98 can be applied to the reference points 116 on the transducer arrays, and then, the transducer array fasteners 114 can be connected to the complementary transducer array fasteners 98. Then, one of the support panels 66 may be bent to overlay a particular one of the transducer arrays 30 and contact the transducer array fasteners 114. The transducer array fasteners 114 may be provided with a bonding material, such as an adhesive or a cohesive to connect the transducer array fasteners 114 to the support panel 66. Once the transducer array fasteners 114 are connected to the support panel 66, then the transducer array fasteners 114 may be removed from the complementary transducer array fasteners 98 and the support panel 66 may be released so as to move the support panel 66 away from the particular transducer array 30. This procedure registers the transducer array fasteners 114 with the pattern and location of the complementary transducer array fasteners 98 to permit reapplication of the transducer array 30 in the same location on the patient 54. This procedure can then be repeated for each of the support panels 66.

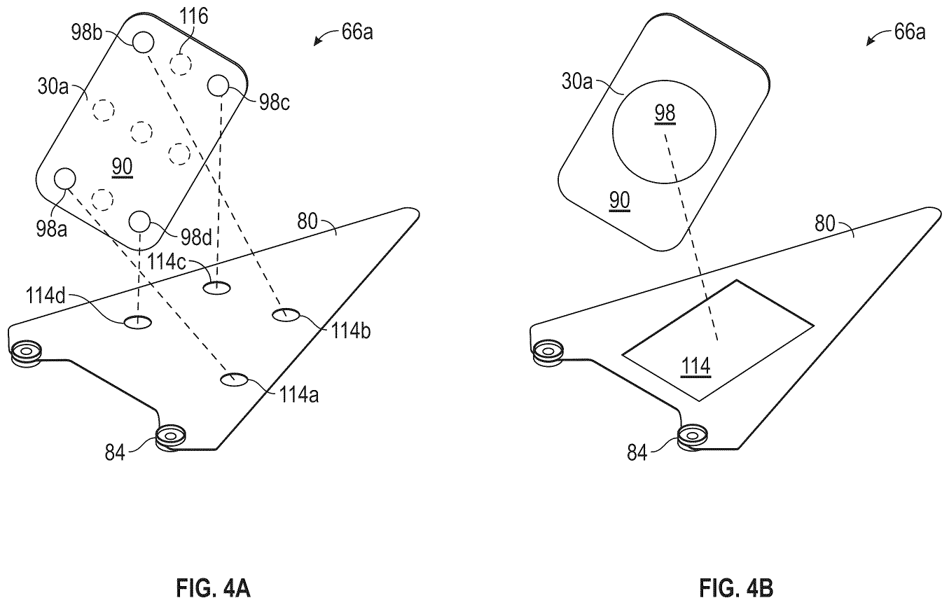

Referring now to FIG. 5, shown therein is a cross-sectional view of an exemplary embodiment of the first transducer array 30a attached to the first support panel 66a and applied to the patient 54. As shown in FIG. 5, the first transducer array 30a has the attaching components 94, shown as a first attaching component 94a, a second attaching component 94b, and a third attaching component 94c, and the complementary transducer array fasteners 98, shown as a first complementary transducer array fastener 98a and a second complementary transducer array fastener 98b. Further, the first support panel 66a has the transducer array fasteners 114, shown as a first transducer array fastener 114a and a second transducer array fastener 114b.

As further shown in FIG. 5, the first support panel 66a is shown with the first transducer array fastener 114a and the second transducer array fastener 114b attached to the first complementary transducer array fastener 98a and the second complementary transducer array fastener 98b, respectively, and the first transducer array 30a is shown with the first attaching component 94a, the second attaching component 94b, and the third attaching component 94c attached to the patient 54.

Also shown in FIG. 5 is one or more fastener force, shown as a first fastener force 118a and a second fastener force 118b (collectively, the “fastener forces 118”), exerted on the first transducer array 30a when a force is applied to the first support panel 66a to pull the first support panel 66a away from the patient 54, thereby causing the transducer array fasteners 114 to disengage from the complementary transducer array fasteners 98. Further shown is one or more attaching force, shown as a first attaching force 122a, a second attaching force 122b, and a third attaching force 122c (collectively, the “attaching forces 122”) exerted on the first transducer array 30a when the force is applied to the first support panel 66a due to the attaching components 94. In other words, as the force is applied to the first support panel 66a to pull the first support panel 66a away from the patient 54, the force is transferred through the transducer array fasteners 114 attached to the complementary transducer array fasteners 98, the first transducer array 30a, and the attaching components 94 to the patient 54. As the force applied is increased, the fastener forces 118 and the attaching forces 122 also increase.

As depicted, the attaching forces 122 are greater than the fastener forces 118. Thus, when the force applied to pull the first support panel 66a away from the patient 54 is approximate to the fastener forces 118, the transducer array fasteners 114 will disengage from the complementary transducer array fasteners 98 while the first transducer array 30a maintains placement on the patient 54, due to the attaching forces 122 exceeding the fastener forces 118. In this manner, the apparatus 50 may be used as an applicator for applying one or more of the transducer arrays 30 to the patient 54, preferably one at a time and in a sequential manner.

In one embodiment, the attaching components 94 may be selected such that the attaching forces 122 of the attaching components 94 when applied to the patient 54 are greater than that fastener forces 118 between the transducer array fasteners 114 and the complementary transducer array fasteners 98. In one embodiment, a sum of the attaching forces 122 is greater than a sum of the fastener forces 118. In one embodiment, the transducer array fasteners 114 and the complementary transducer array fastener 98 are chosen such that the fastener forces 118 are lesser than the attaching forces 122. In one embodiment, the attaching components 94 may be adhesives or cohesives. A cohesive is a bonding material that works on the principle of cohesion force. Cohesion force is a force that attracts molecules of the same substance. For example, the attaching components 94 may be adhesives attached to the first transducer array 30a during manufacturing or during use.

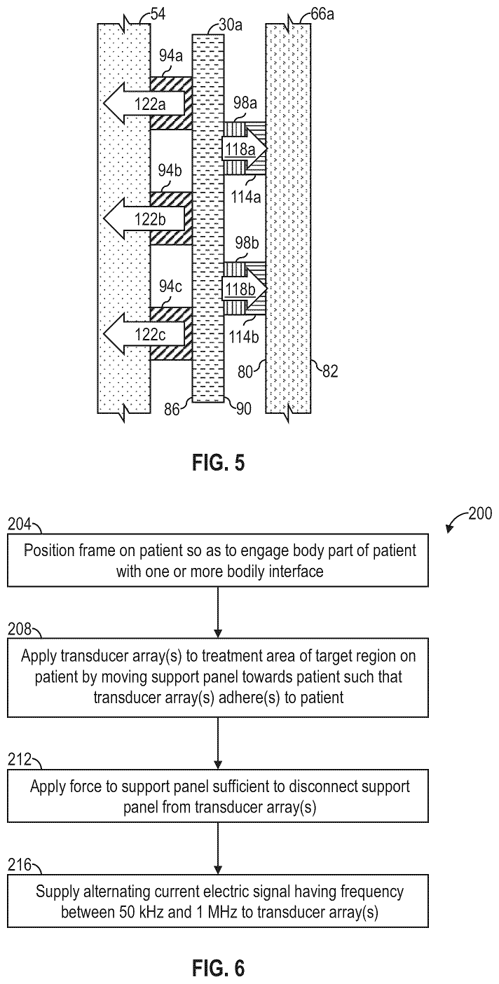

Referring now to FIG. 6, shown therein is a method 200 of using the system 10 as described herein. As shown in FIG. 6, the method 200 generally comprises: attaching the first transducer array 30a having the complementary transducer array fasteners 98 to the first support panel 66a having the transducer array fasteners 114, attaching the second transducer array 30b having the complementary transducer array fasteners 98 to the second support panel 66b having the transducer array fasteners 114 (as shown in FIG. 2), attaching the third transducer array 30c having the complementary transducer array fasteners 98 to the third support panel 66c having the transducer array fasteners 114 (as shown in FIG. 2) and attaching the fourth transducer array 30d having the complementary transducer array fasteners 98 to the fourth support panel 66d having the transducer array fasteners 114 (as shown in FIG. 2). Once the transducer arrays 30 are attached to the support panels 66, the support panels may be attached to the frame 62 as described above.

Then, the frame 62 of the apparatus 50 may be positioned on the patient 54 so as to engage the body part 74 of the patient 54 with one or more bodily interface 70, the frame 62 having the support panel 66 attached to the frame 62 and with the one or more transducer array 30 attached to the support panel 66 (step 204); for each support panel 66 supporting one or more of the transducer arrays 30, applying the one or more transducer array 30 to the treatment area 58 of a target region within the patient 54 by moving the support panel 66 towards the patient 54 such that the one or more transducer array 30 adheres to the patient 54 (step 208); for each support panel 66 supporting one or more of the transducer arrays 30, applying force to the support panel 66 sufficient to disconnect the support panel 66 from the one or more transducer array 30 (step 212); removing the frame 62 of the apparatus 50 from the patient 54; and supplying an alternating current electric signal having a frequency between 50 kHz and 1 MHz to the one or more transducer array 30 (step 216) to provide the TTField to the treatment area 58 of the patient 54.

In some embodiments, applying the force to the support panel 66 is further defined as removing the support panel 66 from the patient 54 such that one or more transducer array 30 adheres to the patient 54. In some embodiments, wherein the one or more bodily interface 70 includes the nose interface 70a, the left ear interface 70b, and the right ear interface 70c, and positioning the frame 62 of the apparatus 50 on the patient 54 is further defined as: positioning a central portion 100 of the frame 62 on the patient 54 such that the nose interface 70a engages the patient's nose 74a; adjusting the left portion 102 of the frame 62 such that the left ear interface 70b engages the patient's left ear 74b; and adjusting the right portion 104 of the frame 62 such that the right ear interface 70c engages the patient's right ear 74c.

Referring now to FIGS. 7A-7C, shown therein is another exemplary embodiment of the apparatus 50 shown in FIG. 2 (hereinafter, the “torso apparatus 50a”). In the embodiment shown in FIGS. 7A-7C, the torso apparatus 50a is configured to be worn on a torso of the patient 54.

The torso apparatus 50a comprises another exemplary embodiment of the frame 62 (hereinafter, the “torso frame 62a”) and the support panels 66, such as the first support panel 66a, the second support panel 66b, the third support panel 66c, and the fourth support panel 66d shown in FIG. 7A, and a fifth support panel 66e, a sixth support panel 66f, a seventh support panel 66g, and an eighth support panel 66h shown in FIG. 7B. The torso frame 62a is similar in construction and operation as the frame 62, with the exception that the torso frame 62a is sized and shaped to be positioned in a predetermined location on the torso of the patient 54.

In some embodiments, each of the support panels 66 temporarily supports one or more of the transducer arrays 30 attached thereto, such as the first transducer array 30a, the second transducer array 30b, the third transducer array 30c, and the fourth transducer array 30d shown in FIG. 7A, and a fifth transducer array 30e, a sixth transducer array 30f, a seventh transducer array 30g, and an eighth transducer array 30h shown in FIG. 7B.

As shown in FIGS. 7A-7C, the torso frame 62a includes bodily interfaces 70. The bodily interfaces 70 include a shoulder interface 70d configured to engage the patient's shoulders 74d, a right armpit interface 70e configured to engage the patient's right armpit 74e, and a left armpit interface 70f configured to engage the patient's left armpit 74f. In some embodiments, the torso frame 62a may be rigid such that, when the bodily interfaces 70 are engaging the body parts of the patient 54, the torso frame 62a is maintained in a fixed position without substantial deflection when supporting the support panels 66. In some such implementations, the torso frame 62a may be constructed using a plastic material, such as polycarbonate, polypropylene, polyethylene, or polyvinyl chloride, for example.

The torso frame 62a may have the outer surface 76, a skin-facing surface (not shown), and the support panel fasteners 78 in one or more location on the outer surface 76 adjacent to the treatment area 58 when the torso apparatus 50a is positioned on the patient 54. For purposes of clarity, only one of the support panel fasteners 78 is labeled with a reference character. In the embodiment shown, the torso frame 62a includes twenty of the support panel fasteners 78 spatially disposed on the outer surface 76 of the frame 62 and configured to be positioned adjacent to the treatment area 58 when the apparatus 50 is positioned on the patient 54. However, it should be understood that the torso frame 62a may include a number of the support panel fasteners 78 greater or less than twenty.

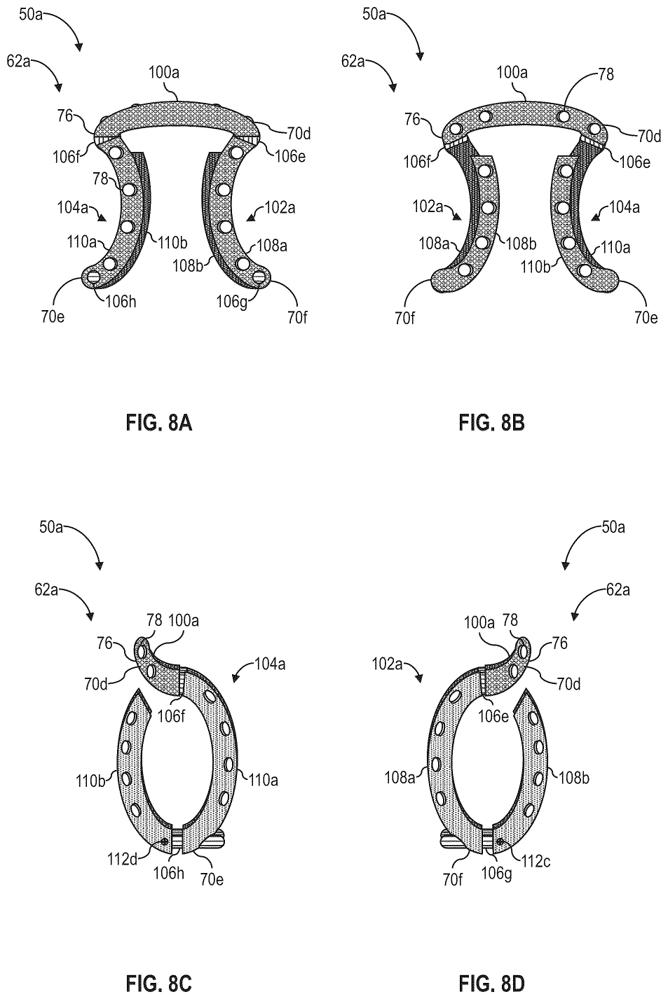

Referring now to FIGS. 8A-8D, shown therein is another exemplary embodiment of the torso frame 62a of the torso apparatus 50a shown in FIGS. 7A-7C. In the embodiment shown, the torso frame 62a comprises a central portion 100a configured to extend over an upper back behind the neck of the patient 54, a left portion 102a configured to extend around a left shoulder of the patient 54, and a right portion 104a configured to extend around a right shoulder of the patient 54.

The central portion 100a may have one or more connection point, such as a left connection point 106e and a right connection point 106f spaced a distance laterally from the left connection point 106e. The left portion 102a may be connected to the central portion 100a at the left connection point 106e and extend downwardly from the central portion 100a such that the left portion 102a is movable (e.g., pivotable) about the left connection point 106e, and the right portion 104a may be connected to the central portion 100a at the right connection point 106f and extend downwardly from the central portion 100a such that the right portion 104a is movable (e.g., pivotable) about the right connection point 106f.

In some embodiments, the left portion 102a of the torso frame 62a further comprises a left front portion 108a configured to extend across a left portion of a chest of the patient 54 and a left rear portion 108b configured to extend across a left portion of a back of the patient 54. In such embodiments, the left portion 102a may have a left rear connection point 106g disposed between the left front portion 108a and the left rear portion 108b. The left rear portion 108b may slidingly engage the left rear connection point 106g such that the left rear portion 108b may slide forwards and backwards to accommodate different sizes of the patient 54.

Similarly, in some embodiments, the right portion 104a of the torso frame 62a further comprises a right front portion 110a configured to extend across a right portion of the chest of the patient 54 and a right rear portion 110b configured to extend across a right portion of the back of the patient 54. In such embodiments, the right portion 104a may have a right rear connection point 106h disposed between the right front portion 110a and the right rear portion 110b. The right rear portion 110b may slidingly engage the right rear connection point 106h such that the right rear portion 110b may slide forwards and backwards to accommodate different sizes of the patient 54.

In some embodiments, one or more of the left rear connection point 106g and the right rear connection point 106h may have one or more locking mechanism, such as a left locking mechanism 112c and a right locking mechanism 112d, to maintain the left rear portion 108b and the right rear portion 110b in place when the left locking mechanism 112c and the right locking mechanism 112d are locked.

Although the embodiment of the torso frame 62a shown is constructed of different parts that are movable relative to each other to be able to adjust the size of the torso frame 62a, it should be understood that the torso frame 62a may be formed as a unitary device designed to fit a particular size of the patient 54.

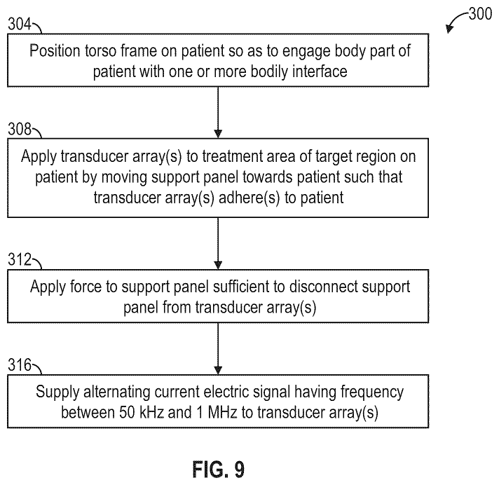

Referring now to FIG. 9, shown therein is a method 300 of using the system 10 including the torso apparatus 50a as described herein. As shown in FIG. 9, the method 300 generally comprises: positioning the torso frame 62a of the torso apparatus 50a on the patient 54 so as to engage the body parts 74 of the patient 54 (e.g., the patient's shoulders 74d, the patient's right armpit 74e, and the patient's left armpit 74f) with the bodily interfaces 70 (e.g., the shoulder interface 70d, the right armpit interface 70e, and the left armpit interface 70f), the torso frame 62a having the support panels 66 (e.g., the first support panel 66a, the second support panel 66b, the third support panel 66c, the fourth support panel 66d, the fifth support panel 66e, the sixth support panel 66f, the seventh support panel 66g, and the eighth support panel 66h) attached to the torso frame 62a and having the transducer arrays 30 (e.g., the first transducer array 30a, the second transducer array 30b, the third transducer array 30c, the fourth transducer array 30d, the fifth transducer array 30e, the sixth transducer array 30f, the seventh transducer array 30g, and the eighth transducer array 30h) attached to the support panels 66 (step 304); for each support panel 66 supporting one or more transducer array 30, applying the one the one or more transducer array 30 to the treatment area 58 of a target region within the patient 54 by moving the support panel 66 towards the patient 54 such that the one or more transducer array 30 adheres to the patient 54 (step 308); for each support panel 66 supporting one or more transducer array 30, applying force to the support panel 66 sufficient to disconnect the support panel 66 from the one or more transducer array 30 (step 312); and supplying an alternating current electric signal having a frequency between 50 kHz and 1 MHz to the one or more transducer array 30 (step 316) to provide the TTField to the treatment area 58 of the patient 54.

In some embodiments, the method 300 further comprises, prior to positioning the torso frame 62a of the torso apparatus 50a on the patient 54 (step 304): attaching the first transducer array 30a having the complementary transducer array fasteners 98 to the first support panel 66a having the transducer array fasteners 114 (as shown in FIG. 7A), attaching the second transducer array 30b having the complementary transducer array fasteners 98 to the second support panel 66b having the transducer array fasteners 114 (as shown in FIG. 7A), attaching the third transducer array 30c having the complementary transducer array fasteners 98 to the third support panel 66c having the transducer array fasteners 114 (as shown in FIG. 7A), attaching the fourth transducer array 30d having the complementary transducer array fasteners 98 to the fourth support panel 66d having the transducer array fasteners 114 (as shown in FIG. 7A), attaching the fifth transducer array 30e having the complementary transducer array fasteners 98 to the fifth support panel 66e having the transducer array fasteners 114 (as shown in FIG. 7B), attaching the sixth transducer array 30f having the complementary transducer array fasteners 98 to the sixth support panel 66f having the transducer array fasteners 114 (as shown in FIG. 7B), attaching the seventh transducer array 30g having the complementary transducer array fasteners 98 to the seventh support panel 66g having the transducer array fasteners 114 (as shown in FIG. 7B), and attaching the eighth transducer array 30h having the complementary transducer array fasteners 98 to the eighth support panel 66h having the transducer array fasteners 114 (as shown in FIG. 7B).

In some embodiments, positioning the torso frame 62a of the torso apparatus 50a on the patient 54 (step 304) further comprises the steps of: positioning the central portion 100a of the torso frame 62a on the upper back of the patient 54 behind the neck such that the shoulder interface 70d engages the patient's shoulders 74d; positioning the left portion 102a of the torso frame 62a such that the left portion 102a extends around the left shoulder of the patient 54; and positioning the right portion 104a of the torso frame 62a such that the right portion 104a extends around the right shoulder of the patient 54.

In some embodiments, positioning the left portion 102a of the torso frame 62a further comprises the steps of: pivoting the left front portion 108a of the torso frame 62a about the left connection point 106e such that the left front portion 108a extends across the left portion of the chest of the patient 54; and adjusting the left rear portion 108b of the torso frame 62a such that the left rear portion 108b extends across the left portion of the back of the patient 54 and the left armpit interface 70f engages the patient's left armpit 74f.

In some embodiments, positioning the right portion 104a of the torso frame 62a further comprises the steps of: pivoting the right front portion 110a of the torso frame 62a about the right connection point 106f such that the right front portion 110a extends across the right portion of the chest of the patient 54; and adjusting the right rear portion 110b of the torso frame 62a such that the right rear portion 110b extends across the right portion of the back of the patient 54 and the right armpit interface 70e engages the patient's right armpit 74e.

In some embodiments, the method 300 further comprises, subsequent to applying force to the support panel 66 (step 312): removing the torso frame 62a of the torso apparatus 50a from the patient 54.

### Non-Limiting Illustrative Embodiments of the Inventive Concept(s)

The following is a number list of non-limiting illustrative embodiments of the inventive concepts disclosed herein:

Illustrative embodiment 1. An apparatus, comprising: a frame having one or more bodily interface configured to engage a body part of a patient and one or more support panel fastener; a support panel sized and dimensioned to cover at least a portion of a treatment area of a target region and comprising one or more complementary support panel fastener configured to attach to the one or more support panel fastener; a transducer array having a skin-facing surface and an opposed support panel surface and comprising a complementary transducer array fastener positioned on the opposed support panel surface and an attaching component positioned on the skin-facing surface; and one or more transducer array fastener connected to the support panel and configured to attach to the complementary transducer array fastener so as to support the transducer array within at least the portion of the treatment area, wherein the one or more transducer array fastener is separable from the complementary transducer array fastener with a first force, the attaching component when attached to the patient is separable from the patient with a second force greater than the first force, and application of the first force to the transducer array in a direction away from the patient does not cause separation of the transducer array from the patient.

Illustrative embodiment 2. The apparatus of illustrative embodiment 1, wherein the support panel is a first support panel, the portion is a first portion, the one or more complementary support panel fastener is one or more first complementary support panel fastener, the transducer array is a first transducer array, the skin-facing surface is a first skin-facing surface, the opposed support panel surface is a first opposed support panel surface, the complementary transducer array fastener is a first complementary transducer array fastener, the attaching component is a first attaching component, the one or more transducer array fastener is one or more first transducer array fastener, and the apparatus further comprises: a second support panel sized and dimensioned to cover at least a second portion of the treatment area and comprising one or more second complementary support panel fastener configured to attach to the one or more support panel fastener; a second transducer array having a second skin-facing surface and a second opposed support panel surface and comprising a second complementary transducer array fastener positioned on the second opposed support panel surface and a second attaching component positioned on the second skin-facing surface; and one or more second transducer array fastener connected to the second support panel and configured to attach to the second complementary transducer array fastener so as to support the second transducer array within at least the second portion of the treatment area, wherein the one or more second transducer array fastener is separable from the second complementary transducer array fastener with a third force, the second attaching component when attached to the patient is separable from the patient with a fourth force greater than the third force, and application of the third force to the second transducer array in a direction away from the patient does not cause separation of the second transducer array from the patient.

Illustrative embodiment 3. The apparatus of illustrative embodiment 2, wherein the first portion and the second portion partially coincide.

Illustrative embodiment 4. The apparatus of illustrative embodiment 2, wherein the first portion and the second portion substantially coincide.

Illustrative embodiment 5. The apparatus of illustrative embodiment 1, wherein the one or more bodily interface includes a nose interface configured to engage a patient's nose, a left ear interface configured to engage a patient's left ear, and a right ear interface configured to engage a patient's right ear.

Illustrative embodiment 6. The apparatus of illustrative embodiment 5, wherein the frame comprises a central portion, a left portion, and a right portion, the central portion having a first connection point and a second connection point, the left portion connected to the central portion at the first connection point such that the left portion is pivotable about the first connection point, the right portion connected to the central portion at the second connection point such that the right portion is pivotable about the second connection point, the nose interface connected to the central portion, the left ear interface connected to the left portion, and the right ear interface connected to the right portion.

Illustrative embodiment 7. The apparatus of Illustrative embodiment 1, wherein the one or more bodily interface includes a first bodily interface configured to engage a first body part of the patient, a second bodily interface configured to engage a second body part of the patient, and a third bodily interface configured to engage a third body part of the patient.

Illustrative embodiment 8. The apparatus of Illustrative embodiment 7, wherein the frame comprises a central portion, a left portion, and a right portion, the central portion having a first connection point and a second connection point, the left portion connected to the central portion at the first connection point such that the left portion is pivotable about the first connection point, the right portion connected to the central portion at the second connection point such that the right portion is pivotable about the second connection point, the first bodily interface connected to the central portion, the second bodily interface connected to the left portion, and the third bodily interface connected to the right portion.

Illustrative embodiment 9. The apparatus of illustrative embodiment 1, wherein the one or more support panel fastener is further defined as a plurality of spatially disposed support panel fasteners configured to be positioned adjacent to the treatment area.

Illustrative embodiment 10. The apparatus of illustrative embodiment 1, wherein the transducer array is attached to the support panel via the complementary transducer array fastener engaging the one or more transducer array fastener, the transducer array operable to receive a TTField signal and generate a TTField based on the TTField signal.

Illustrative embodiment 11. The apparatus of illustrative embodiment 1, wherein one of the one or more support panel fastener and the one or more complementary support panel fastener is a snap member and the other of the one or more support panel fastener and the one or more complementary support panel fastener is a receiving member, the snap member configured to engage the receiving member.

Illustrative embodiment 12. The apparatus of illustrative embodiment 1, wherein one of the one or more transducer array fastener and the complementary transducer array fastener is a hook member and the other of the one or more transducer array fastener and the complementary transducer array fastener is a loop member, the hook member configured to engage the loop member.

Illustrative embodiment 13. A system, comprising: a first transducer array configured to be electrically connected to an electric field generator and operable to receive a TTField signal and generate a TTField based on the TTField signal, the first transducer array having a first complementary transducer array fastener and a first attaching component; a second transducer array configured to be electrically connected to an electric field generator and operable to receive a TTField signal and generate a TTField based on the TTField signal, the second transducer array having a second complementary transducer array fastener and a second attaching component; and an apparatus, comprising: a frame having one or more bodily interface configured to engage a body part of a patient and one or more support panel fastener; a first support panel sized and dimensioned to cover at least a first portion of a treatment area of a target region and comprising one or more first complementary support panel fastener configured to attach to the one or more support panel fastener; a second support panel sized and dimensioned to cover at least a second portion of the treatment area and comprising one or more second complementary support panel fastener configured to attach to the one or more support panel fastener; one or more first transducer array fastener connected to the first support panel and configured to attach to the first complementary transducer array fastener so as to support the first transducer array within at least the first portion of the treatment area; and one or more second transducer array fastener connected to the second support panel and configured to attach to the second complementary transducer array fastener so as to support the second transducer array within at least the second portion of the treatment area; and wherein the one or more first transducer array fastener and the one or more second transducer array fastener are separable from the first complementary transducer array fastener and the second complementary transducer array fastener with a first force, the first attaching component and the second attaching component when attached to the patient are separable from the patient with a second force greater than the first force, and application of the first force to the first transducer array and the second transducer array in a direction away from the patient does not cause separation of the first transducer array and the second transducer array from the patient.

Illustrative embodiment 14. The system of illustrative embodiment 13, wherein the one or more bodily interface includes a nose interface configured to engage a patient's nose, a left ear interface configured to engage a patient's left ear, and a right ear interface configured to engage a patient's right ear.

Illustrative embodiment 15. The system of illustrative embodiment 14, wherein the frame comprises a central portion, a left portion, and a right portion, the central portion having a first connection point and a second connection point, the left portion connected to the central portion at the first connection point such that the left portion is pivotable about the first connection point, the right portion connected to the central portion at the second connection point such that the right portion is pivotable about the second connection point, the nose interface connected to the central portion, the left ear interface connected to the left portion, and the right ear interface is connected to the right portion.

Illustrative Embodiment 16. The system of illustrative embodiment 13, wherein the one or more bodily interface includes a first bodily interface configured to engage a first body part of the patient, a second bodily interface configured to engage a second body part of the patient, and a third bodily interface configured to engage a third body part of the patient.

Illustrative Embodiment 17. The system of illustrative embodiment 16, wherein the frame comprises a central portion, a left portion, and a right portion, the central portion having a first connection point and a second connection point, the left portion connected to the central portion at the first connection point such that the left portion is pivotable about the first connection point, the right portion connected to the central portion at the second connection point such that the right portion is pivotable about the second connection point, the first bodily interface connected to the central portion, the second bodily interface connected to the left portion, and the third bodily interface connected to the right portion.

Illustrative embodiment 18. The system of illustrative embodiment 13, wherein the one or more support panel fastener is further defined as a plurality of support panel fasteners configured to be positioned adjacent to the treatment area.

Illustrative embodiment 19. The system of illustrative embodiment 13, wherein the first transducer array is attached to the first support panel via the first complementary transducer array fastener engaging the one or more first transducer array fastener, the first transducer array operable to receive a TTField signal and generate a TTField based on the TTField signal.

Illustrative embodiment 20. The system of illustrative embodiment 13, wherein one of the one or more support panel fastener and the one or more first complementary support panel fastener is a snap member and the other of the one or more support panel fastener and the one or more first complementary support panel fastener is a receiving member, the snap member configured to engage the receiving member.

Illustrative embodiment 21. The system of illustrative embodiment 13, wherein one of the one or more first transducer array fastener and the first complementary transducer array fastener is a hook member and the other of the one or more first transducer array fastener and the first complementary transducer array fastener is a loop member, the hook member configured to engage the loop member.

Illustrative embodiment 22. A method, comprising: positioning a frame of an apparatus on a patient so as to engage a body part of the patient with one or more bodily interface, the frame having a support panel attached to the frame and one or more transducer array attached to the support panel; applying the one or more transducer array to a treatment area of a target region within the patient by moving the support panel towards the patient such that the one or more transducer array adheres to the patient; applying force to the support panel sufficient to disconnect the support panel from the one or more transducer array; and supplying an alternating current electric signal having a frequency between 50 kHz and 1 MHz to the one or more transducer array.

Illustrative embodiment 23. The method of illustrative embodiment 22, wherein applying the force to the support panel is further defined as removing the support panel from the patient such that one or more transducer array adheres to the patient.

Illustrative embodiment 24. The method of illustrative embodiment 22, wherein the one or more bodily interface includes a nose interface, a left ear interface, and a right ear interface, and positioning the frame of the apparatus on the patient is further defined as: positioning a central portion of the frame on the patient such that the nose interface engages a patient's nose; adjusting a left portion of the frame such that the left ear interface engages a patient's left ear; and adjusting a right portion of the frame such that the right ear interface engages a patient's right ear.

25. The method of claim 22, wherein the one or more bodily interface includes a first bodily interface, a second bodily interface, and a third bodily interface, and positioning the frame of the apparatus on the patient is further defined as:
positioning a central portion of the frame on the patient such that the first bodily interface engages a first body part of the patient;
adjusting a left portion of the frame such that the second bodily interface engages a second body part of the patient; and
adjusting a right portion of the frame such that the third bodily interface engages a third body part of the patient.

Illustrative embodiment 26. An apparatus, comprising: a rigid frame having one or more bodily interface configured to engage a body part of a patient and one or more support panel fastener; a flexible support panel comprising one or more complementary support panel fastener configured to attach to the one or more support panel fastener, the flexible support panel being movable to cover at least a portion of a treatment area of a target region within the patient; a transducer array having a skin-facing surface and an opposed support panel surface and comprising a complementary transducer array fastener positioned on the opposed support panel surface and an attaching component positioned on the skin-facing surface; and one or more transducer array fastener connected to the flexible support panel and configured to attach to the complementary transducer array fastener so as to support the transducer array within at least the portion of the treatment area when the flexible support panel is moved to cover at least the portion of the treatment area.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features are recited in the claims and/or disclosed in the specification, these combinations are not intended to limit the disclosure. In fact, many of these features may be combined in ways not specifically recited in the claims and/or disclosed in the specification. Although each dependent claim listed below may directly depend on only one other claim, the disclosure includes each dependent claim in combination with every other claim in the claim set.

No element, act, or instruction used in the present application should be construed as critical or essential to the invention unless explicitly described as such outside of the preferred embodiment. Further, the phrase “based on” is intended to mean “based at least in part on” unless explicitly stated otherwise.

What is claimed is:

1. An apparatus, comprising:
a frame having one or more bodily interface configured to engage a body part of a patient and one or more support panel fastener;
a support panel sized and dimensioned to cover at least a portion of a treatment area of a target region and comprising one or more complementary support panel fastener configured to attach to the one or more support panel fastener;
a transducer array having a skin-facing surface and an opposed support panel surface and comprising a complementary transducer array fastener positioned on the opposed support panel surface and an attaching component positioned on the skin-facing surface; and
one or more transducer array fastener connected to the support panel and configured to attach to the complementary transducer array fastener so as to support the transducer array within at least the portion of the treatment area, wherein the one or more transducer array fastener is separable from the complementary transducer array fastener with a first force, the attaching component when attached to the patient is separable from the patient with a second force greater than the first force, and application of the first force to the transducer array in a direction away from the patient does not cause separation of the transducer array from the patient.

2. The apparatus of claim 1, wherein the support panel is a first support panel, the portion is a first portion, the one or more complementary support panel fastener is one or more first complementary support panel fastener, the transducer array is a first transducer array, the skin-facing surface is a first skin-facing surface, the opposed support panel surface is a first opposed support panel surface, the complementary transducer array fastener is a first complementary transducer array fastener, the attaching component is a first attaching component, the one or more transducer array fastener is one or more first transducer array fastener, and the apparatus further comprises:
a second support panel sized and dimensioned to cover at least a second portion of the treatment area and comprising one or more second complementary support panel fastener configured to attach to the one or more support panel fastener;
a second transducer array having a second skin-facing surface and a second opposed support panel surface and comprising a second complementary transducer array fastener positioned on the second opposed support panel surface and a second attaching component positioned on the second skin-facing surface; and
one or more second transducer array fastener connected to the second support panel and configured to attach to the second complementary transducer array fastener so as to support the second transducer array within at least the second portion of the treatment area, wherein the one or more second transducer array fastener is separable from the second complementary transducer array fastener with a third force, the second attaching component when attached to the patient is separable from the patient with a fourth force greater than the third force, and application of the third force to the second transducer array in a direction away from the patient does not cause separation of the second transducer array from the patient.

3. The apparatus of claim 2, wherein the first portion and the second portion partially coincide.

4. The apparatus of claim 2, wherein the first portion and the second portion substantially coincide.

5. The apparatus of claim 1, wherein the one or more bodily interface includes a first bodily interface configured to engage a first body part of the patient, a second bodily interface configured to engage a second body part of the patient, and a third bodily interface configured to engage a third body part of the patient.

6. The apparatus of claim 5, wherein the frame comprises a central portion, a left portion, and a right portion, the central portion having a first connection point and a second connection point, the left portion connected to the central portion at the first connection point such that the left portion is pivotable about the first connection point, the right portion connected to the central portion at the second connection point such that the right portion is pivotable about the second connection point, the first bodily interface connected to the central portion, the second bodily interface connected to the left portion, and the third bodily interface connected to the right portion.

7. The apparatus of claim 1, wherein the one or more support panel fastener is further defined as a plurality of spatially disposed support panel fasteners configured to be positioned adjacent to the treatment area.

8. The apparatus of claim 1, wherein the transducer array is attached to the support panel via the complementary transducer array fastener engaging the one or more transducer array fastener, the transducer array operable to receive a TTField signal and generate a TTField based on the TTField signal.

9. The apparatus of claim 1, wherein one of the one or more support panel fastener and the one or more complementary support panel fastener is a snap member and the other of the one or more support panel fastener and the one or more complementary support panel fastener is a receiving member, the snap member configured to engage the receiving member.

10. The apparatus of claim 1, wherein one of the one or more transducer array fastener and the complementary transducer array fastener is a hook member and the other of the one or more transducer array fastener and the complementary transducer array fastener is a loop member, the hook member configured to engage the loop member.

11. A system, comprising:
a first transducer array configured to be electrically connected to an electric field generator and operable to receive a TTField signal and generate a TTField based on the TTField signal, the first transducer array having a first complementary transducer array fastener and a first attaching component;
a second transducer array configured to be electrically connected to an electric field generator and operable to receive a TTField signal and generate a TTField based on the TTField signal, the second transducer array having a second complementary transducer array fastener and a second attaching component; and
an apparatus, comprising:
a frame having one or more bodily interface configured to engage a body part of a patient and one or more support panel fastener;
a first support panel sized and dimensioned to cover at least a first portion of a treatment area of a target region and comprising one or more first complementary support panel fastener configured to attach to the one or more support panel fastener;
a second support panel sized and dimensioned to cover at least a second portion of the treatment area and comprising one or more second complementary support panel fastener configured to attach to the one or more support panel fastener;
one or more first transducer array fastener connected to the first support panel and configured to attach to the first complementary transducer array fastener so as to support the first transducer array within at least the first portion of the treatment area; and
one or more second transducer array fastener connected to the second support panel and configured to attach to the second complementary transducer array fastener so as to support the second transducer array within at least the second portion of the treatment area; and
wherein the one or more first transducer array fastener and the one or more second transducer array fastener are separable from the first complementary transducer array fastener and the second complementary transducer array fastener with a first force, the first attaching component and the second attaching component when attached to the patient are separable from the patient with a second force greater than the first force, and application of the first force to the first transducer array and the second transducer array in a direction away from the patient does not cause separation of the first transducer array and the second transducer array from the patient.

12. The system of claim 11, wherein the one or more bodily interface includes a first bodily interface configured to engage a first body part of the patient, a second bodily interface configured to engage a second body part of the patient, and a third bodily interface configured to engage a third body part of the patient.

13. The system of claim 12, wherein the frame comprises a central portion, a left portion, and a right portion, the central portion having a first connection point and a second connection point, the left portion connected to the central portion at the first connection point such that the left portion is pivotable about the first connection point, the right portion connected to the central portion at the second connection point such that the right portion is pivotable about the second connection point, the first bodily interface connected to the central portion, the second bodily interface connected to the left portion, and the third bodily interface connected to the right portion.

14. The system of claim 11, wherein the one or more support panel fastener is further defined as a plurality of spatially disposed support panel fasteners configured to be positioned adjacent to the treatment area.

15. The system of claim 11, wherein the first transducer array is attached to the first support panel via the first complementary transducer array fastener engaging the one or more first transducer array fastener.

16. The system of claim 11, wherein one of the one or more support panel fastener and the one or more first complementary support panel fastener is a snap member and the other of the one or more support panel fastener and the one or more first complementary support panel fastener is a receiving member, the snap member configured to engage the receiving member.

17. The system of claim 11, wherein one of the one or more first transducer array fastener and the first complementary transducer array fastener is a hook member and the other of the one or more first transducer array fastener and the first complementary transducer array fastener is a loop member, the hook member configured to engage the loop member.

18. A method, comprising:
positioning a frame of an apparatus on a patient so as to engage a body part of the patient with one or more bodily interface, the frame having a support panel attached to the frame and one or more transducer array attached to the support panel;
applying the one or more transducer array to a treatment area of a target region within the patient by moving the support panel towards the patient such that the one or more transducer array adheres to the patient;
applying force to the support panel sufficient to disconnect the support panel from the one or more transducer array; and
supplying an alternating current electric signal having a frequency between 50 kHz and 1 MHz to the one or more transducer array.

19. The method of claim 18, wherein applying the force to the support panel is further defined as removing the support panel from the patient such that one or more transducer array adheres to the patient.

20. The method of claim 18, wherein the one or more bodily interface includes a first bodily interface, a second bodily interface, and a third bodily interface, and positioning the frame of the apparatus on the patient is further defined as:
positioning a central portion of the frame on the patient such that the first bodily interface engages a first body part of the patient;
adjusting a left portion of the frame such that the second bodily interface engages a second body part of the patient; and
adjusting a right portion of the frame such that the third bodily interface engages a third body part of the patient.
